(19) United States
(12) Patent Application Publication
(10) Pub. No.: US 2006/0116335 A1
(43) Pub. Date: Jun. 1, 2006

(54) Ready-for-complexation composition

(75) Inventors: William T. H. Chang, Taipei (TW); James H. Y. Chen, Taipei (TW); Wen Pin Yeh, Taipei County (TW)

Correspondence Address: Ladas & Parry, 26 West 61st Street, New York, NY, 10023, US

(73) Assignee: Lytone Enterprise, Inc.

(21) Appl. No.: 10/998,257

(22) Filed: Nov. 26, 2004

(51) Int. Cl. A61K 31/724; A61K 31/7048

(52) U.S. Cl. 514/28; 514/58

(57) ABSTRACT

The invention relates to a ready-for-complexation (RFC) composition, comprising a polyene antimycotic and a cyclodextrin or the derivatives thereof. Also disclosed is a method of using the ready-for-complexation (RFC) composition of the invention to improve the solubility of polyene antimycotic.

### FIELD OF THE INVENTION

The present invention relates to a ready-for-complexation (RFC) composition, comprising a polyene antimycotic and a cyclodextrin or the derivatives thereof.

### BACKGROUND OF THE INVENTION

Many polyene antimycotics are known to have antifungal properties useful for treating fungal infections. The polyene antimycotics are basically and uniquely characterized by a large lactone ring which includes a chain of conjugated double bonds, specifically comprising 4, 5, 6 or 7 such linkages, whereby the compounds are correspondingly known as tetraenes, pentaenes, hexaenes and heptaenes and are collectively called polyenes. EP0434943 indicates that the polyene antimycotics have a low or almost absent water solubility, which is a common characteristic of all the polyenes and causes a strong hindrance to diffusion after application.

Natamycin, an example of polyene antimycotic, is provided to illustrate the solubility defects of the polyene antimycotic. Natamycin is a member of the polyene family, and has been used to prevent fungal growth on foods for more than 30 years. It is common in the surface treatment of cheese and sausage and can also be used to prevent spoilage of juice and fermented milk by yeast (N. J. Russell and G. W. Gould, Kluewer Academic/Plenum Publishers, New York, pp. 179-195.) However, due to the amphoteric character of the natamycin, it has a low solubility in most solvents. U.S. Pat. No. 6,156,362 indicates that natamycin is relatively insoluble in water, in which its solubility is of the order 0.005-0.010 weight/weighdditionally; even in solution, natamycin is rather unstable. The low solubility of natamycin also limits its application in food process. An enhanced antimycotic activity could be achieved by improving solubility of natamycin, thus making it more available to the food environment. The solubility of natamycin in water can be increased using alkaline, acidic conditions or organic solvents. However, the dissolved natamycin molecule is sensitive to light, oxygen, or extreme pH value. It is well known that dissolved natamycin would rapidly decompose in water.

Accordingly, it would be desirable to solve the problem of poor solubility and solution stability of polyene antimycotics.

### SUMMARY OF THE INVENTION

An object of the invention is to provide a ready-for-complexation (RFC) composition, comprising a polyene antimycotic and a cyclodextrin or the derivatives thereof.

Another object of the invention is to provide a method of using the ready-for-complexation (RFC) composition of the invention to improve the solubility of polyene antimycotics, comprising dissolving the ready-for-complexation composition in water or buffer solution to form a cyclodextrin-polyene antimycotic inclusion complex.

Another object of the invention is to provide a method of using a ready-for-complexation composition for postharvest treatment, comprising providing said composition and applying said composition to postharvest agricultural products, said composition comprises polyene antimycotic and a cyclodextrin or the derivatives thereof.

Another further object of the invention is to provide a method of using a ready-for-complexation composition in culture substrate against fungal infection, comprising providing said composition and applying said composition to a culture substrate, said composition comprises polyene antimycotic and a cyclodextrin or the derivatives thereof.

### DETAILED DESCRIPTION OF THE INVENTION

The present invention provides a ready-for-complexation (RFC) composition, comprising a polyene antimycotic and a cyclodextrin or the derivatives thereof.

According to the invention, the polyene antimycotics used in the ready-for-complexation (RFC) composition of the invention are a group of macrocyclic polyketides that interact with membrane sterols and are, therefore, active against fungi but not bacteria. The macrolide rings of polyene antimycotics are larger than those of standard 14- or 16-membered nonpolyene macrolides. The latter rings include a chromophore of conjugated double bonds, which are the characteristic polyene structure. According to the invention, the polyene antimycotic refers to the polyene macrolides and their derivatives. The polyene macrolide derivatives of the invention comprise a main polyene macrolide backbone derived from any of a variety of polyene macrolides. Examples of these polyene macrolides include, but not limited to, natamycin, amphotericin B, aureofacin, candicidin, candidin, levorin, mycoheptin, nystatin, partricin A, partricin B, perimycin, pimaricin, polyfungin, rimocidin and trichomycin.

Natamycin is one preferred embodiment of polyene antimycotic of the invention. Natamycin is a commonly used polyene antimycotic in preventing fungal growth on foods. Natamycin is a creamy white, odorless, tasteless, especially insoluble crystalline amphoteric powder. The natamycin suitable for use in the invention is a known and commercially available yeast and mold inhibitor that has been used to prevent the growth of yeasts and molds in various products. Natamycin also refers to other names such as pimaricin, antibiotic A 5283, tennecetin, CL 12625, Mycophyt, Myprozine, Natacyn and Pimafucin, all of which are collectively referred to as “natamycin” for the purposes of the invention. According to the invention, natamycin also includes any compounds having substantially the same chemical structure as natamycin, e.g., compounds produced by chemical synthesis or biotechnology, provided such compounds have essentially the same mold and yeast inhibition properties. Natamycin, such as those from Gist-Brocades Food Ingredients, Inc. of King of Prussia, Pa. (DELVOCID.RTM) and Cultor Food Science Inc., Roseville, Calif. (NATAMAX.RTM), is commercially available.

According to the invention, the cyclodextrins used in the ready-for-complexation (RFC) composition of the invention are a group of structurally related saccharides which are formed by enzymatic cyclization of starch by a group of amylases termed glycosyltransferases. The most common naturally occurring cyclodextrins are α-cyclodextrin, β-cyclodextrin and γ-cyclodextrin consisting of 6, 7 and 8glucopyranose units, respectively. The most notable feature of cyclodextrin is their ability to form solid inclusion complexes (host-guest complexes) with a very wide range of solid, liquid and gaseous compounds by a phenomenon of molecular complexation. In these complexes, a guest molecule is held within the cavity of the cyclodextrin host molecule. The term “guest” is used to refer to the compound which is trapped and complexed within the cyclodextrin molecule. Cyclodextrins are cyclic oligosaccharides, consisting of (α-1,4)-linked α-D-glucopyranose units, with a somewhat lipophilic central cavity and a hydrophilic outer surface. The materials such as natamycin to be complexed are trapped within the cavity of the cyclodextrin molecules and held there through a number of different binding mechanisms. According to the invention, appropriate cyclodextrin derivatives can also be used to complex with the natamycin. The term “cyclodextrin derivative” refers to modified cyclodextrin, branched cyclodextrin and their mixtures. According to the invention, cyclodextrin derivatives include, but not limited to, the hydroxypropyl derivatives of α, β and γ-cyclodextrin, sulfoalkylether cyclodextrins such as sulfobutylether β-cyclodextrin, alkylated cyclodextrins such as the randomly methylated β-cyclodextrin, and various branched cyclodextrins such as glucosyl- and maltosyl-β-cyclodextrin.

According to the invention, the ready-for-complexation (RFC) composition is prepared by mixing cyclodextrin or the derivatives thereof with the polyene antimycotics. Also, the molar ration of the cyclodextrin to the polyene antimycotic ranges from 0.05:1 to 5:1. Preferably, the molar ratio ranges from 0.5:1 to 4.:1. More preferably, the molar ratio ranges from 1:1 to 4:1.

The present invention provides a method of using the ready-for-complexation (RFC) composition of the invention to improve the solubility of polyene antimycotics, comprising dissolving the ready-for-complexation composition in water or buffer solution to form a cyclodextrin-polyene antimycotic inclusion complex. Preferably, the concentration of RFC composition in the solution ranges from 1,000 to 30,000 ppm. More preferably, the concentration ranges from 1,500 to 15,000 ppm. The invention further provides a a method of using the ready-for-complexation (RFC) composition of the invention to improve the solubility of polyene antimycotics and a method of using a ready-for-complexation composition in culture substrate against fungal infection, which methods comprise providing said composition and applying said composition to postharvest agricultural products and a culture substrate, respectively.

The ready-for-complexation composition of the invention can effectively solve the low solubility problem of the polyene antimycotics. Particularly, the water solubility of the polyene antimycotics can increase 3 to 7 folds after the ready-for-complexation composition is solved in water. Advantageously, the RFC premix offers significant cost benefit for its effective antimycotic activity at low dosage. Moreover, the described RFC composition of the invention can be used for the treatment of food and agriculture products by methods known as dipping, spraying or dosing in liquid products.

Although, there are many trials for improving the antimycotic activity of polyene and cyclodextrin complexation is also a well-known skill for enhancing the water solubility of hydrophobic compounds. The present invention enlarges the scope of application of polyene antimycotics. For example, the RFC composition of the invention can be used for the treatment of postharvest agricultural products and culture substrate. Preferably, the agricultural products comprises but not limited to vegetables, fruits and meats.

The following examples further illustrate the present invention, but are not intended to limit the scope of the present invention. The modifications and substitutions known to those skilled in the art are still within the scope and spirit of the present invention.

### EXAMPLE

### Example 1

### Preparation of Ready-for-Complexation (RFC) Composition

The example describes the preparation of RFC composition of the invention. β-cyclodextrin (β-CD) was mixed with the polyene antimycotic powder in a molar ratio from 1:1 to 4:1 by hand or mixing devices. The resultant samples were stored at low temperature before use.

### Example 2

### Natamycin Solubility of the RFC composition in Different Molar Ratios of Cyclodextrin to Natamycin

The RFC compositions were prepared by mixing commercial natamycin (87%) withβ-CD(98%) in different molar ratios. A comparison on the solubility of natamycin was made for the RFC compositions. The solubility of natamycin in water is determined by UV Spectrophotometer at 332.6 nm. The results are summarized in Table 1below.

TABLE 1

| | Molar ratio (β-CD:natamycin) | | | | |
|---|---|---|---|---|---|
| | 0:1 | 1.1 | 2.1 | 3:1 | 4:1 |
| Solubility(ppm), 25° C. | 45 | 120 | 215 | 265 | 310 |

As shown in the above results, the solubility of natamycin is proportional to the β-CD amounts in RFC.

### Example 3

### Evaluation of the Antimycotic Activity of RFC Composition

The freshly prepared spore suspension (c.a. 10⁶/ml)of Aspergillus niger (CRCC 30506) was inoculated on MRS agar plates containing different concentrations of natamycin. The diameter of the colonies of Aspergillus niger was measured after being incubated for 7days at 24° C. As shown in Table 2 below, RFC compositions have a lower MIC value than commercial natamycin in a proper range of molar ratio ofβ-CD and natamycin. The results of Table 2 also indicate that the RFC mixture show higher antimycotic activity than conventional natamycin because of the improved solubility.

TABLE 2

| | Commercial | RFC Mixtures |
|---|---|---|
| Natamycin | natamycin | Molar ratio = 1:1˜1:4 |
| Source | (50%) | (β-CD:natamycin) |
| MIC (ppm) | 1.25˜1.5 | <1.0 |

### Example 4

### Application of RFC composition for Postharvest Treatment

This example describes the potential application of RFC composition for postharvest treatment.

To simulate the fruit body infected by fungi, freshly harvested Ponkans were wounded by sterilized nail and immersed in a solution containing penicillium spores (10⁷ cfu/ml) for 3 minutes. After been treated with penicillium spores solution, all Ponkans fruit were been dried at room temperature for 5 hours and divided into four test groups (27 for each) at random. Each group was treated with 0, 0.1, 0.05 and 0.01% of RFC solution (molar ratio of β-CD and natamycin is 4:1) for 5 minutes to investigate the protection effect of RFC. As the results shown in Table 3, RFC treatment groups have lower percentage of decay by penicillium.

TABLE 3

| | Percentage of decay | | | |
|---|---|---|---|---|
| Days after | RFC concentration (%) | | | |
| inoculation | 0(Control) | 0.01 | 0.05 | 0.1 |
| 1 | 0 | 0 | 0 | 0 |
| 5 | 33.3 | 0 | 0 | 0 |
| 11 | 72.2 | 5.6 | 0 | 0 |
| 15 | 100 | 11.1 | 0 | 0 |
| 30 | 100 | 11.1 | 11.1 | 0 |
| 60 | 100 | 16.7 | 11.1 | 11.1 |

### Example 5

### Antifungal activity of RFC composition on Fusarium oxysporum f. sp. radicis-lycopersici of Tomato Seedling

Tomato seeds were sown in mixed substrate (pearl, peat moss, and vermiculite) at 72 cells of plug tray. Tomato seedling at the four-leaf stage were carefully transplanted into 35 cells of plug tray which contain culture substrate with 4×10⁶ cfu/g of FORL, a tomato isolated Fusarium oxysporum f. sp. radicis-lycopersici. After planting, culture substrate was watered with 0.04% of RFC (molar ratio of β-CD and natamycin is 4:1) solution or tap water (control) 3 times in the following period of 30 days. As shown in Table 4, culture substrate treated with 0.04% of RFC is effective in reducing the incidence of disease during tomato seedling growth.

TABLE 4

| | Percentage of incidence | |
|---|---|---|
| Days after | RFC concentration (%) | |
| inoculation | 0(Control) | 0.04 |
| 1 | 0 | 0 |
| 5 | 0 | 0 |
| 10 | 0 | 0 |
| 15 | 3.33 | 0 |
| 30 | 13.33 | 3.33 |
| 40 | 83.33 | 23.33 |

What is claimed is:

1. A ready-for-complexation (RFC) composition, comprising a polyene antimycotic and a cyclodextrin or the derivatives thereof.

2. The ready-for-complexation composition of claim 1, wherein the polyene antimycotic is selected from the group consisting of natamycin, amphotericin B, aureofacin, candicidin, candidin, levorin, mycoheptin, nystatin, partricin A, partricin B, perimycin, pimaricin, polyfungin, rimocidin and trichomycin.

3. The ready-for-complexation composition of claim 2, wherein the polyene antimycotic is natamycin.

4. The ready-for-complexation composition of claim 1, wherein the cyclodextrin is selected from the group consisting of α-cyclodextrin, β-cyclodextrin and γ-cyclodextrin.

5. The ready-for-complexation composition of claim 4, wherein the cyclodextrin is β-cyclodextrin.

6. The ready-for-complexation composition of claim 1, wherein the cyclodextrin or the derivative is selected from the group consisting of hydroxypropyl derivatives of α, β and γ-cyclodextrin, sulfoalkylether cyclodextrins, alkylated cyclodextrins and branched cyclodextrins.

7. The ready-for-complexation composition of claim 6, wherein the branched cyclodextrin is glucosyl-β-cyclodextrin or maltosyl-β-cyclodextrin.

8. The ready-for-complexation composition of claim 1, wherein the molar ration of the cyclodextrin to the polyene antimycotic ranges from 0.05:1 to 5:1.

9. The ready-for-complexation composition of claim 8, wherein the molar ration of the cyclodextrin to the polyene antimycotic ranges from 0.5:1 to 4.:1.

10. The ready-for-complexation composition of claim 9, wherein the molar ration of the cyclodextrin to the polyene antimycotic ranges from 1:1 to 4.:1.

11. A method of using a ready-for-complexation composition to improve the solubility of polyene antimycotics, comprising providing said ready-for-complexation composition and solving said composition in water or buffer solution to form a cyclodextrin-polyene antimycotic inclusion complex;
wherein said composition comprises polyene antimycotic and a cyclodextrin or the derivatives thereof.

12. The method of claim 11, wherein the concentration of the ready-for-complexation composition in the solution ranges from 1,000 to 30,000 ppm.

13. The method of claim 11, wherein the concentration of the ready-for-complexation composition in the solution ranges from 1,500 to 15,000 ppm.

14. A method of using a ready-for-complexation composition for postharvest treatment, comprising providing said composition and applying said composition to postharvest agricultural products, said composition comprises polyene antimycotic and a cyclodextrin or the derivatives thereof.

15. A method of using a ready-for-complexation composition in culture substrate against fungal infection, comprising providing said composition and applying said composition to a culture substrate, said composition comprises polyene antimycotic and a cyclodextrin or the derivatives thereof.
